(19) United States
(12) Patent Application Publication
(10) Pub. No.: US 2024/0315712 A1
(43) Pub. Date: Sep. 26, 2024

(54) ASPIRATION THROMBECTOMY SYSTEM

(71) Applicant: MicroVention, Inc., Aliso Viejo, CA (US)

(72) Inventors: Joseph Rye, Aliso Viejo, CA (US); Tao Lin, Aliso Viejo, CA (US); Xiang Simon Han, Aliso Viejo, CA (US); Sarri Alsunna, Aliso Viejo, CA (US); Hussain S. Rangwala, Aliso Viejo, CA (US); Hari Narayanan Raman, Aliso Viejo, CA (US)

(73) Assignee: MicroVention, Inc., Aliso Viejo, CA (US)

(21) Appl. No.: 18/692,118

(22) PCT Filed: Sep. 22, 2022
(86) PCT No.: PCT/US2022/076861

§ 371 Date: Mar. 14, 2024

Related U.S. Application Data
(60) Provisional application No. 63/247,146, filed on Sep. 22, 2021.

(51) Int. Cl. A61B 17/22; A61B 17/00

(52) U.S. Cl. CPC A61B 17/22; A61B 2017/00292; A61B 2017/22079; A61B 2217/005

(57) ABSTRACT

Aspiration thrombectomy devices and methods wherein an aspiration cycle includes a positive displacement segment to facilitate clot removal, a pump cartridge to facilitate tube management and an aspiration device for providing a vacuum surge. Disclosed herein is an aspiration thrombectomy system which relies on air or inert gas, rather than liquid, to provide both static and dynamic suction level profiles for safely and efficiently retrieving and removing from the body a thrombus. In one example embodiment, two or more pressure sources may be configured to apply two or more suction levels to an aspiration catheter. Valves may connect conduits between the pressure sources and the aspiration catheter, with the valves being selectively opened or closed to control which pressure level is applied to the catheter.

### RELATED APPLICATIONS

This application claims priority to U.S. Provisional Application Ser. No. 63/247,146 filed Sep. 22, 2021 entitled Aspiration Thrombectomy Devices and Methods, which is hereby incorporated herein by reference in its entirety.

### BACKGROUND OF THE INVENTION

Described herein are systems, devices, and methods used to perform thrombus aspiration by utilizing suction pressure applied to an aspiration catheter. When performing thrombus aspiration, the thrombus may often become stuck in the catheter. More specifically, the thrombus may become stuck at the entrance to the catheter, particularly if the thrombus is of a greater size than the entry opening of the catheter.

There are disadvantages presented by standard, liquid-primed aspiration pumps that may be utilized with various aspiration catheters. Such liquid-primed pumps may require additional steps for setup which can cost valuable time, such as the need for setting up relief tubing and/or a liquid source such as a saline bag.

There is a need for an aspiration thrombectomy system which relies on air or inert gas, rather than liquid, and which is capable of both static suction levels for retrieving a thrombus and dynamic or surge suction levels for breaking up the thrombus if it becomes stuck in an aspiration catheter.

### SUMMARY OF THE INVENTION

Disclosed herein is an aspiration thrombectomy system which relies on air or inert gas, rather than liquid, to provide both static and dynamic suction level profiles for safely and efficiently retrieving and removing from the body a thrombus.

In one example embodiment, two or more pressure sources may be configured to apply two or more suction levels to an aspiration catheter. Valves may connect conduits between the pressure sources and the aspiration catheter, with the valves being selectively opened or closed to control which pressure level is applied to the catheter.

A steady pressure level may be applied to the catheter for retrieving the thrombus, and a dynamic pressure level may be applied to the catheter for breaking up the thrombus if it becomes stuck in the catheter.

In one example embodiment, an aspiration module may be fluidly connected between an aspiration pump and an aspiration catheter.

In one example embodiment, a disposable and replaceable cartridge may be removably positioned within an associated housing. A pair of conduits, each in communication with a different pressure source applying a different pressure, may be routed through the cartridge. Hammers may be actuated by an actuator, such as a solenoid, to selectively seal and release each of the conduits to control the suction level applied to the catheter.

In one example embodiment, a cylinder may include an internal plunger configured to selectively apply a surge pressure level when needed to break up a thrombus that has become stuck in a catheter. The plunger may include a valve which opens (e.g., hingedly, axially, or rotatably) upon adjustment of the plunger from a first position towards a second position. The plunger may be composed of a paramagnetic material such that energizing of a coil may adjust the plunger towards the second position. A biasing member such as a spring may bias the plunger towards the first position.

In one example embodiment, an enclosure may enclose a sliding mechanism which is adjustable between a first position and a second position. In the first position, the sliding mechanism may seal a first conduit connected between a first pressure source and a catheter. In the second position, the sliding mechanism may seal a second conduit connected between a second pressure source and the same catheter. An actuator such as a solenoid may be operable to adjust the sliding mechanism between its positions. A biasing member such a spring may bias the sliding mechanism towards its first position.

In one example embodiment, the sliding mechanism may be split between two separate portions, separated by a spring, to prevent both conduits from being either opened or closed simultaneously.

A method of performing thrombus aspiration may comprise applying a first pressure level to an aspiration catheter when retrieving a clot and, if the clot becomes stuck in the catheter, applying a second pressure level to break up the clot.

Another method of performing thrombus aspiration may comprise applying both a steady vacuum profile and a dynamic vacuum profile, and switching between the two, as needed to both retrieve a clot and break up the clot if it becomes stuck.

Another method of performing thrombus aspiration may comprise applying a surge vacuum pressure to the catheter if the clot becomes stuck in the catheter.

Another method of performing thrombus aspiration may comprise syncing different pressure levels with the cardiac rhythm of a patient to ensure that systolic pressure of the cardiac rhythm does not carry the clot away from the catheter.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 is a block diagram of an aspiration thrombectomy system according to an example embodiment.

FIG. 2A is a block diagram of an aspiration thrombectomy system in a first operational state according to an example embodiment.

FIG. 2B is a block diagram of an aspiration thrombectomy system in a second operational state according to an example embodiment.

FIG. 3 is an exploded view of a cartridge of an aspiration thrombectomy system according to an example embodiment.

FIG. 4A is a first perspective view of a cartridge of an aspiration thrombectomy system according to an example embodiment.

FIG. 4B is a second perspective view of a cartridge of an aspiration thrombectomy system according to an example embodiment.

FIG. 5 is an upper perspective view of a housing and cartridge of an aspiration thrombectomy system according to an example embodiment.

FIG. 5 is a view of a cartridge assembly in relation to a receptacle with solenoids installed.

FIG. 6 is a sectional view of an aspiration thrombectomy system in a first operational state according to an example embodiment.

FIG. 7 is a sectional view of an aspiration thrombectomy system in a second operational state according to an example embodiment.

FIG. 8 is a sectional view of an aspiration thrombectomy system in a third operational state according to an example embodiment.

FIG. 9 is a first perspective view of an aspiration thrombectomy system according to an example embodiment.

FIG. 10 is a second perspective view of an aspiration thrombectomy system according to an example embodiment.

FIG. 11 is a perspective view illustrating connection of a catheter to a pair of conduits of an aspiration thrombectomy system according to an example embodiment.

FIG. 12 is a sectional view of an aspiration thrombectomy system according to an example embodiment.

FIG. 13A is a perspective view of an aspiration thrombectomy system according to an example embodiment.

FIG. 13B is a perspective view of an aspiration thrombectomy system according to an example embodiment.

FIG. 14 is a graph illustrating a first vacuum pressure profile of an aspiration thrombectomy system according to an example embodiment.

FIG. 15 is a graph illustrating a second vacuum pressure profile of an aspiration thrombectomy system according to an example embodiment.

FIG. 16 is a graph illustrating a vacuum pressure profile in alignment with a patient's cardiac rhythm in accordance with an example embodiment.

### DETAILED DESCRIPTION

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

For the purposes of the terminology described below, the terms clot, thrombus, embolus, and obstruction can be used synonymously.

For the purposes of this specification, use of the terms “about”, “around”, or “approximately” when referring to a value may be understood to mean within 5% of the stated value (either greater or lesser), inclusive.

The present invention may comprise an aspiration thrombectomy system which is capable of selectively applying different levels of pressure against a catheter for the purpose of retrieving a clot from a patient and depositing the clot outside of the patient's body, such as within a container such as a canister.

The aspiration thrombectomy system may apply a static pressure profile, in which a steady, constant suction level if applied to the catheter, or a dynamic pressure profile, in which differing suction levels are applied to the catheter to produce a “jack hammer” effect and thus aid in breaking up clots that may become stuck in the catheter. The system may alternate between such the static profile and the dynamic pressure profile as needed.

The aspiration thrombectomy system may include one or more pressure sources configured to apply different levels of pressure against a catheter. The catheter may be connected to the pressure source(s) by one or more valves, with the number of valves matching the number of pressure levels to be applied. For example, in a system in which two different pressure levels may be applied, two valves may be connected between a catheter and two different pressure sources.

Multiple pressure sources may be integrated into a single pump console. Pressure levels may be alternated as needed to either apply a steady, constant suction level, such as for retrieving a clot, or a dynamic, changing suction level, such as for freeing breaking up a clot that has become stuck in the catheter. A container, such as a canister, may be fluidly connected to the catheter to eventually collect the clot after it has been removed from the patient's body.

The aspiration thrombectomy system may utilize a cartridge which may be removably inserted within a corresponding housing. The cartridge may be disposable such that, after use, it may be disposed of and replaced by a new cartridge. A plurality of conduits may be routed through the cartridge, with each conduit being fluidly connected to a different pressure source applying a different level of pressure and with both conduits converging to be connected to the catheter.

The cartridge may include openings, such as slots, through which each conduit may be routed. Each opening may include an enlarged gap so as to allow a hammer or other mechanism to selectively engage with an exposed portion of each conduit so as to seal the conduit, such as by pinching or pressing against the conduit. An actuator such as a solenoid may be attached to each hammer so as to control advancing or retracting the hammer and thus sealing or unsealing a respective conduit.

The aspiration thrombectomy system may utilize a cylinder including an internal plunger that may be adjusted between at least two positions. The plunger may include a valve which is hingedly attached to selectively seal a passageway extending through the plunger. An actuator such as a coil may be operable to adjust the plunger in a first direction so as to open the valve. A biasing member such as a spring may be operable to adjust the plunger in a second direction back to its starting position absent force from the actuator. Adjusting the plunger and opening the valve may be operable to apply a surge pressure level to a connected catheter to aid in breaking up a clot that has become stuck in the catheter.

The aspiration thrombectomy system may be configured to facilitate a non-linear, dynamic thrombectomy using a novel approach wherein a disposable module, provided in a terminally sterilized state as part of an aspiration tubing kit, provides the ability to aspirate a clot using either a linear vacuum pressure profile or various non-linear profiles.

Such a module may comprise an enclosure including internal components which, working together, may effectuate both the linear vacuum pressure profile and various non-linear pressure profiles. A slide mechanism may be adjustable between a first position in which the slide mechanism seals a first conduit and a second position in which the slide mechanism seals a second conduit, with the first conduit being connected between the catheter and a first pressure source and the second conduit being connected between the catheter and a second pressure source.

An actuator such as a solenoid may be utilized to adjust the slide mechanism towards a second position, with a biasing member such as a spring functioning to revert the slide mechanism back to its first, starting position absent application of any force from the actuator. The slide mechanism may be split between two separate portions, separated by a slide spring, to ensure that both conduits are never opened or sealed simultaneously.

The aspiration thrombectomy system may be configured to alternate between static and dynamic vacuum levels being applied to the catheter as needed. Static vacuum levels may be optimal for retrieving a clot, while dynamic vacuum levels may be optimal for breaking up the clot if it becomes stuck in the catheter, such as within a distal opening of the catheter. The vacuum levels may be synced with a cardiac rhythm of a patient such that a first vacuum level is applied between beats and a second vacuum level is applied in sync with the beats to ensure that systolic pressure of the cardiac rhythm does not carry the clot away from the catheter.

The aspiration thrombectomy system may utilize air, rather than liquid, as a relief medium. Such a configuration may allow for easier setup than systems using a liquid medium, such as by eliminating the need for relief tubing and/or a liquid source such as a saline bag. It has been found that air may be utilized without travelling into the catheter itself; with the air instead travelling back into the vacuum pump. By surging air instead of fluid, the same “jack hammer” effect may be produced without the use of liquids.

The aspiration thrombectomy system may be biased towards a certain pressure level so as to act as a failsafe. Generally, the system may be biased towards a higher pressure level such that, if power is lost or the actuator fails, the system may continue to apply suction to the catheter.

Specific example embodiments are described further below. However, it should be understood that any of the features from any of the embodiments can be mixed and matched with each other in any combination. Hence, the present invention should not be restricted to only these embodiments, but any broader combination thereof.

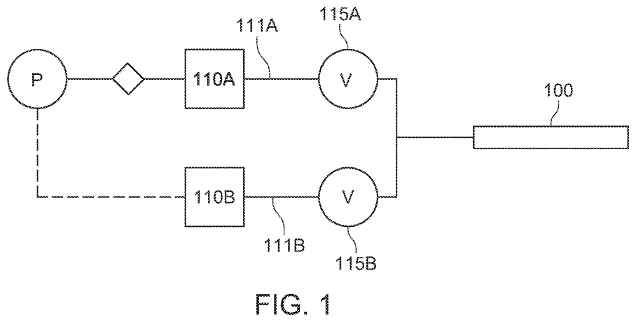

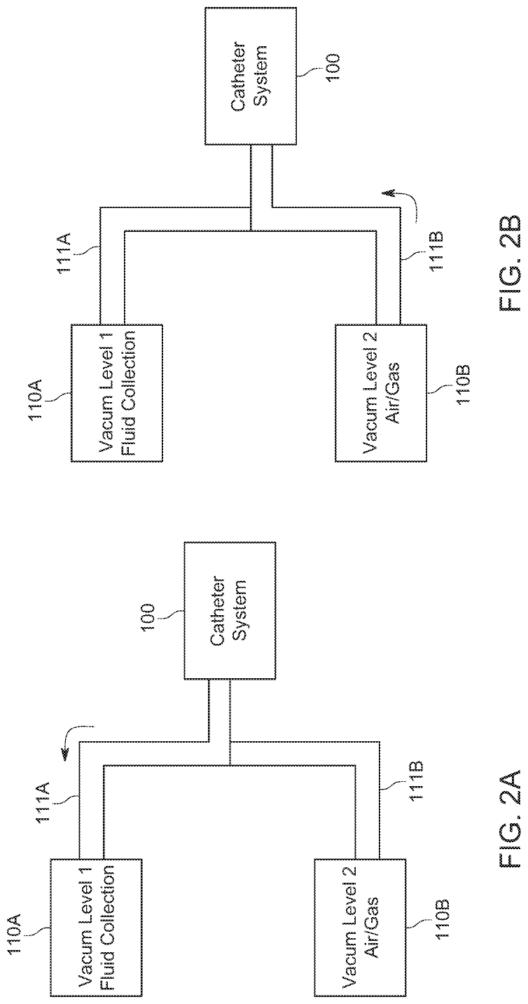

FIGS. 1-2B illustrate a first example embodiment in which two different pressure levels may be selectively applied to an aspiration catheter 100. As shown in FIG. 1, a pair of pressure sources 110A, 110B may be fluidly connected to the aspiration catheter 100 by a pair of valves 115A, 115B. In some embodiments, the pair of pressure sources 110A, 110B may be comprised of a single pump P. However, in other embodiments, each pressure source 110A, 110B may include its own pump.

In an example embodiment, a process may be used to operate an unprimed system having two volumes with known pressure levels. The first pressure source 110A may have apply a first pressure and the second pressure source 110B may apply a second pressure. In some example embodiments, the second pressure applied by the second pressure source 110B may be greater than the first pressure applied by the first pressure source 110A.

By way of example and without limitation, the first pressure may be between approximately 508-762 mmHg below ambient pressure and the second pressure may be between approximately 0-508 mmHg below ambient pressure. The first pressure may be used, e.g., to collect fluid and/or a clot. The first pressure source 110A may thus include a collection canister or other container for collecting the clot.

The second pressure may be used, e.g., to increase the pressure in the aspiration catheter 100, which may be useful for various function such as, for example, breaking up a clot that has become stuck at the distal end of the aspiration catheter 100. The medium of both of the pressure sources 110A, 110B may be air or an inert gas.

Continuing to reference FIG. 1, it can be seen that the first pressure source 110A may be connected to the catheter 100 by a first conduit 111A including a first valve 115A and the second pressure source 110B may be connected to the catheter 100 by a second conduit 111B including a second valve 115B. The types of valves 115A, 115B may vary widely in different embodiments. Thus, the systems and methods described herein should not be construed as being limited to any particular type of valve 115A, 115B, as a wide range of valves 115A, 115B known in the art to selectively seal off a pressure source 110A, 110B from a catheter 100, including the various valves 115A, 115B described herein, may be utilized.

FIGS. 2A and 2B illustrate two configurations which may be used to alternate pressure applied to the catheter 100. In the configuration illustrated in FIG. 2A, it can be seen that the first valve 115A is open and the second valve 115B is closed such that the first pressure is applied to the catheter 100. Such a configuration may be utilized for collecting a clot, which may be drawn through the first valve 115A to be collected within a canister or other container that is integral with, or connected to, the first pressure source 110A.

In the configuration illustrated in FIG. 2B, it can be seen that the first valve 115A is closed and the second valve 115B is opened such that the second pressure is applied to the catheter 100. Such a configuration may be utilized to raise the pressure of the fluid in the catheter 100 by, for example, allowing air or an inert gas at a known pressure to enter the system and thus raise the pressure of the fluid in the catheter 100. The duration of time during which the second valve 115B is opened may be controlled so that air or other gases do not exit the catheter 100.

In some example embodiments, the two valves 115A, 115B may never be opened simultaneously. Thus, in such embodiments, either the first valve 115A is opened and the second valve 115B is closed, or the first valve 115B is closed and the second valve 115B is opened, with no state in which both valves 115A, 115B are simultaneously opened. In some embodiments, however, there may be short instances of time in which both valves 115A, 115B are opened as discussed herein.

The example embodiment shown in FIGS. 1-2B may be switchable between at least two operational modes. A first operational mode may apply static aspiration, with the first valve 115A being left open. The first operational mode may result in application of a constant vacuum level (e.g., between about 508-762 mmHg below ambient pressure) being applied to the catheter 100. If the catheter 100 is occluded by a blood clot in such an operational mode, the system may exert a constant force on the blood clot.

A second operational mode may apply dynamic aspiration by cycling between the two pressure sources 110A, 110B. The second operational mode may be desirable when a blood clot has occluded the catheter 100. In the second operational mode, the valves 115A, 115B may be alternatively opened and closed so as to sequentially apply the first pressure or the second pressure to the catheter 100. During the first half of the cycle, as shown in FIG. 2A, the first pressure source 110A may be connected to the catheter to apply the first pressure, thus creating a large force on the blood clot. During the second half of the cycle, as shown in FIG. 2B, the second pressure source 110B may be connected to the catheter 100 to apply an even higher pressure and reduce force on the blood clot.

Repeating this cycle may cause the pressure changes at tip of the catheter 100 to serve as a “jack hammer” against the clot to “fatigue” the clot and thus break it up. Since the tip of the catheter 100 cannot move back and forth, the “fatigue” is achieved by moving the viscoelastic clot against the proximal part of the device and causing it to stretch and then fit inside the inner diameter of the catheter 100. This may increase the probability of capturing the blood clot in a canister or other container, such as a container which is integral with or connected to the first pressure source 110A.

Although not shown in the figures, it should be appreciated that additional operational modes may be utilized in some example embodiments. For example, additional valves may be utilized so as to introduce a volume of air into the system for modulating the resulting “jack hammer” effect, since the air or inert gas could be compressed in a third conduit.

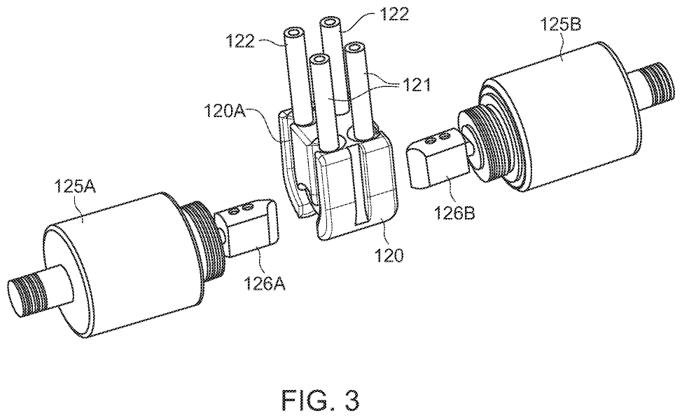

FIGS. 3-5 illustrate an example embodiment in which pinch valves are replaced with solenoids fitted with hammers suited for efficiently activating the pinch tubing. As best shown in FIG. 3, such an example embodiment may comprise a cartridge 120 through which a pair of conduits 121, 122 may be routed, with each of the conduits 121, 122 being connected between a pressure source 110A, 110B and a catheter 100. Solenoid-activated hammers 126A, 126B may be operable to selectively and alternatively pinch the conduits 121, 122 to control a pressure level applied to the catheter 100.

In the example embodiment shown in FIG. 3, it can be seen that each of the conduits 121, 122 may be arranged in a U-shaped configuration, however, other arrangements may be utilized in some embodiments. Generally, a first conduit 121 may extend in a U-shaped arrangement through a first side of the cartridge 120 and a second conduit 122 may extend in a U-shaped arrangement through a second side of the cartridge 120, parallel to the first conduit 121.

Each of the conduits 121, 122 may be in communication, such as by a fluid connection, with both the catheter 100 and at least one pressure source 110A, 110B. More specifically, the first conduit 121 may be connected at a first end 121A to a first pressure source 110A and at a second end 121B to the catheter 100. Similarly, the second conduit 122 may be connected at a first end 122A to a second pressure source 110B and at a second end 122B to the catheter 100. Thus, the first conduit 121 may be connected between the first pressure source 110A and the catheter 100 and the second conduit 122 may be connected between the second pressure source 110B and the catheter 100.

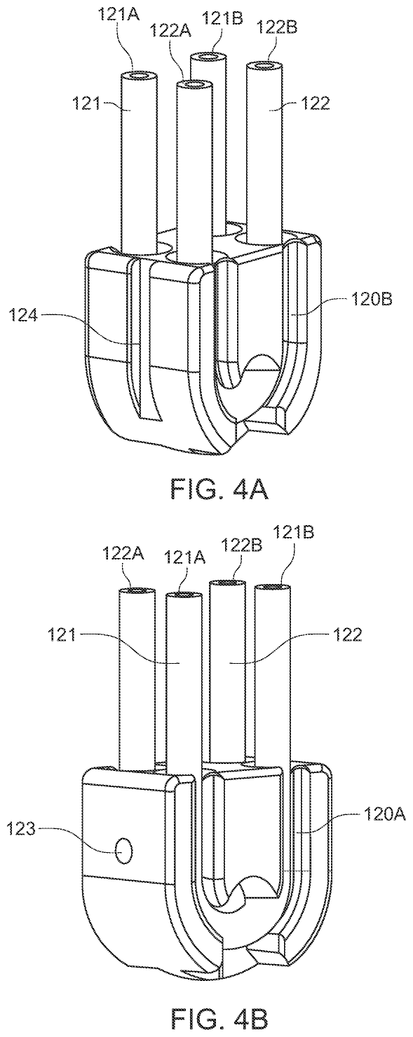

With reference to FIGS. 3-4B, it can be seen that an example embodiment of a cartridge 120 may comprise a substantially U-shaped configuration, with a curved lower end and a flat upper end. Such a configuration may aid in easily inserting and removing the cartridge 120 into and out of an associated cavity 130A of a housing 130 as discussed in more detail below. It should be appreciated, however, that the shape, size, and configuration of the cartridge 120 may vary in different embodiments to accommodate, e.g., different types of conduits 121,122, different numbers of conduits 121, 122, different types of housings 130, and the like.

As best shown in FIGS. 4A and 4B, the cartridge 120 may include cartridge openings 120A, 120B through which the conduits 121, 122 may be routed. More specifically, it can be seen that a first end of the cartridge 120, shown in FIG. 4B, may include a first opening 120A comprised of a curved, generally U-shaped slot. Similarly, it can be seen that a second end of the cartridge 120, shown in FIG. 4A, may include a second opening 120B comprised of a curved, generally U-shaped slot. A medial segment of the first conduit 121 may thus be routed through the first opening 120A and a medial segment of the second conduit 122 may thus be routed through the second opening 120B.

Continuing to reference FIGS. 4A and 4B, it can be seen that each of the openings 120A, 120B may include an enlarged gap which may be sized, oriented, and positioned to receive a corresponding hammer 126A, 126B to selectively and releasably seal the respective conduit 121, 122 positioned therein. Although the enlarged gap of each opening 120A, 120B is illustrated as being positioned near the bottom of the cartridge 120, it should be appreciated that various other positions may be utilized in different embodiments.

It should be appreciated that a wide range of conduits 121, 122 known in the art may be utilized. Generally, each conduit 121, 122 may comprise an elongated, resilient, flexible or semi-flexible tubular member having a lumen extending between a first end 121A, 122A and a second end 121B, 122B of the conduit 121, 122. In an example embodiment, each conduit 121, 122 may comprise pinch tubing.

As best shown in FIGS. 4A and 4B, the cartridge 120 may include features to aid in properly inserting the cartridge 120 within an associated housing 130. In the illustrated example embodiment, it can be seen that the first side of the cartridge 120 may include a detent receiver 123 (such as an opening) and that the second side of the cartridge 120 may include a key slot 124.

The detent receiver 123 may be configured to engage with a corresponding detent 130B of the housing 130 to releasably secure the cartridge 120 to the housing 130. The key slot 124 may be configured to engage with a corresponding key 130C on the housing 130 to guide the cartridge 120 into the cavity 130A of the housing 130. It should be appreciated, however, that alternative configurations may be utilized in different embodiments.

For example, the positions of the detent receiver 123 and key slot 124 on the cartridge 120 may be reversed; with the detent receiver 123 being on the second side of the cartridge 120 and the key slot 124 being on the first side of the cartridge 120. As another example, the configurations of the guiding/locking features may also be reversed by, for example, having the detent receiver 123 in the housing 130 rather than the cartridge 120, having the detent 130B on the cartridge 120 rather than the housing 130, having the key slot 124 in the housing 130 rather than the cartridge 120, and/or having the key 130C on the cartridge 120 rather than the housing 130.

While the figures illustrate a pair of conduits 121, 122 positioned within the cartridge 120 so as to selectively connect a catheter 100 with one of a pair of pressure sources 110A, 110B, it should be appreciated that additional conduits 121, 122 and/or pressure sources 110A, 110B may be utilized in different embodiments. For example, in some embodiments, three or more conduits 121, 122 may be utilized in connection with three or more pressure sources 110A, 110B. Such a configuration may allow for additional pressure levels to be applied to the catheter 100 as needed.

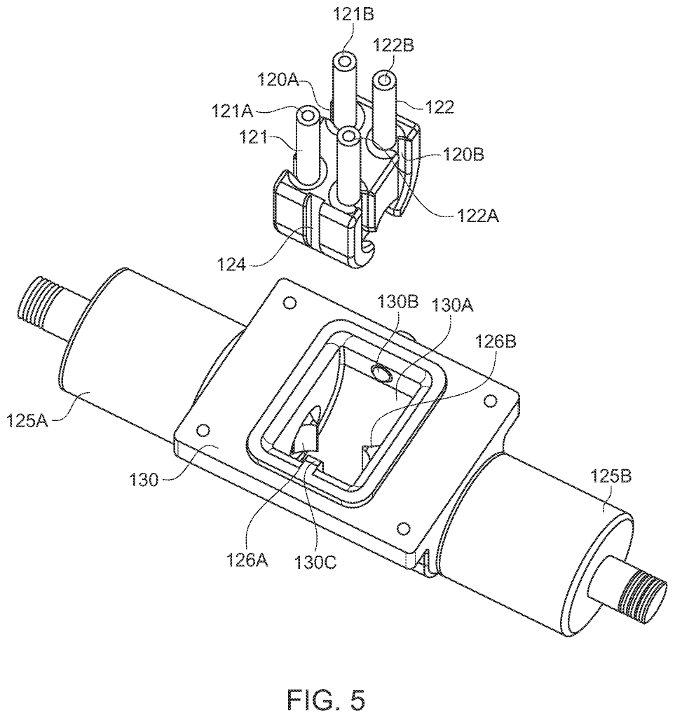

Turning to FIG. 5, it can be seen that the cartridge 120 may be removably inserted within a housing 130. More specifically, the housing 130 may include a cavity 130A into which the cartridge 120 may be removably inserted for use. The shape, size, and positioning of the cavity 130A may vary in different embodiments, and thus should not be construed as limited in scope by the example embodiments shown in the figures. For example, while FIG. 5 illustrates that the cavity 130A may be on the upper end of the housing 130, the cavity 130A may be positioned at various other locations, such as on the sides, of the housing 130 in different embodiments.

Continuing to reference FIG. 5, it can be seen that a pair of solenoids 125A, 125B may be attached to the housing 130. More specifically, it can be seen that a first solenoid 125A may be attached to a first side of the housing 130 and a second solenoid 125B may be attached to a second side of the housing 130. The solenoids 125A, 125B may be fixedly attached, removably attached, or integrally formed with the housing 130 in different embodiments. In the embodiment shown in FIG. 3, it can be seen that each solenoid 125A, 125B may include threading so as to threadably engage with the housing 130. In this manner, the solenoids 125A, 125B may be removed as needed for maintenance or replacement.

It should be appreciated that, while the figures and description refer to “solenoids”, various other types of mechanical, magnetic, electrical, and/or electromagnetic actuators may be utilized to perform the same function as the illustrated solenoids 125A, 125B, and thus the scope should not be construed as limited to solenoids 125A, 125B for actuation of the hammers 126A, 126B. A wide range of actuators known in the art may be utilized instead of or in addition to solenoids 125A, 125B in different embodiments.

As best shown in FIGS. 3 and 5, each of the solenoids 125A, 125B may include at least one hammer 126A, 126B for selectively and releasably engaging with one of the conduits 121, 122. More specifically, it can be seen that the first solenoid 125A may be connected to a first hammer 126A and that the second solenoid 125B may be connected to a second hammer 126B. Each of the hammers 126A, 126B may include a projection adapted to seal a respective conduit 121, 122, such as by pinching the conduit 121, 122. Thus, each of the hammers 126A, 126B may be positioned to be aligned with the enlarged gap of the openings 120A, 120B of the cartridge 120 through which a portion of each conduit 121, 122 may be exposed.

With reference to FIGS. 3-5, the stroke of each solenoid 125A, 125B may be such that, when the solenoid 125A, 125B retracts to open the fluid pathway, the corresponding hammer 126A, 126B moves back far enough to fully relieve the respective conduit 121, 122. With the hammers 126A, 126B out of the way, the cartridge 120 may be easily removed and a new one (with new conduits 121, 122 installed) may be installed just as easily. The cartridge 120 may be provided to the user with the conduits 121, 122 preassembled; thereby eliminating any stretching of the conduits 121, 122 needed to be performed by the user to install the conduits 121, 122 into the valves. This general design may be oriented differently to suit space requirements or to counter other forces such as gravity. This design may also provide a user-friendly and repeatable method for the user to install and remove the tubing kit from the pinch valves in the pump console.

Generally, each solenoid 125A, 125B may be operable to advance or retract an associated hammer 126A, 126B such that each hammer 126A, 126B may seal or release a corresponding conduit 121, 122. Generally, the solenoids 125A, 125B may be alternatively advanced and retracted so as to switch between which pressure source 110A, 110B is applied to the catheter 100. In some instances, such as during brief moments of time, both pressure sources 110A, 110B may be applied to the catheter 100. However, in some embodiments, the system may be configured to prevent both pressure sources 110A, 110B from being simultaneously in communication with the catheter 100.

While the figures illustrate an example embodiment in which a pair of solenoids 125A, 125B and a pair of hammers 126A, 126B are in use with a pair of conduits 121, 122, it should be appreciated that, in other embodiments, additional solenoids 125A, 125B and/or hammers 126A, 126B may be utilized, particularly when additional conduits 121, 122 and/or pressure sources 110A, 110B are utilized. Thus, the scope should not be construed as limited to the example embodiment of the figures in which two solenoids 125A, 125B having two hammers 126A, 126B are utilized to selectively seal two conduits 121, 122 in communication with two pressure sources 110A, 110B. For example, where three or more conduits are in communication with three or more pressure sources, three or more solenoids including three or more hammers may be utilized.

In use, the example embodiment shown in FIGS. 3-5 may be utilized to alternatively apply at least two pressure levels to an aspiration catheter 100. A first pressure level may be utilized for drawing a clot towards and into the catheter 100. A second pressure level may be selectively applied to aid in breaking up the clot in the event it becomes stuck at any point in the catheter 100.

The cartridge 120 may be inserted within the cavity 130A of the housing 130. The key slot 124 of the cartridge 120 may be aligned with the corresponding key 130C within the housing 130, and then the cartridge 120 may be lowered into the cavity 130A. Upon being fully inserted, the detent 130B of the housing 130 will engage with the detent receiver 123 of the cartridge 120, often producing an audible click to indicate to the user that the cartridge 120 has been properly installed.

Before or after the cartridge 120 is inserted within the housing 130, the conduits 121, 122 may be fluidly connected to the pressure sources 110A, 110B and catheter 100. Generally, the first end 121A of the first conduit 121 may be fluidly connected to the first pressure source 110A and the first end 122A of the second conduit 122 may be fluidly connected to the second pressure source 110A. The second ends 121B, 122B of the conduits 121, 122 may be connected to a single catheter 100, such as by a Y-shaped connector or the like.

With the cartridge 120 inserted within the housing 130 and the conduits 121, 122 connected between the pressure sources 110A, 110B and the catheter 100, the system is ready for use. The catheter 100 may be routed to a target location within a patient's body using methods known in the art. Upon reaching the target location, the pressure sources 110A, 110B may be selectively activated or deactivated as needed to aspirate a clot such that the clot may be retrieved, such as within a canister. The solenoids 125A, 125B may be selectively activated such that their respective hammers 126A, 126B seal or release the conduits 121, 122 and thereby control a level of pressure applied to the catheter 100. Upon completion of the process, the cartridge 120 may be removed from the housing 130 and discarded, to be eventually replaced by a different cartridge 120 when needed for further use.

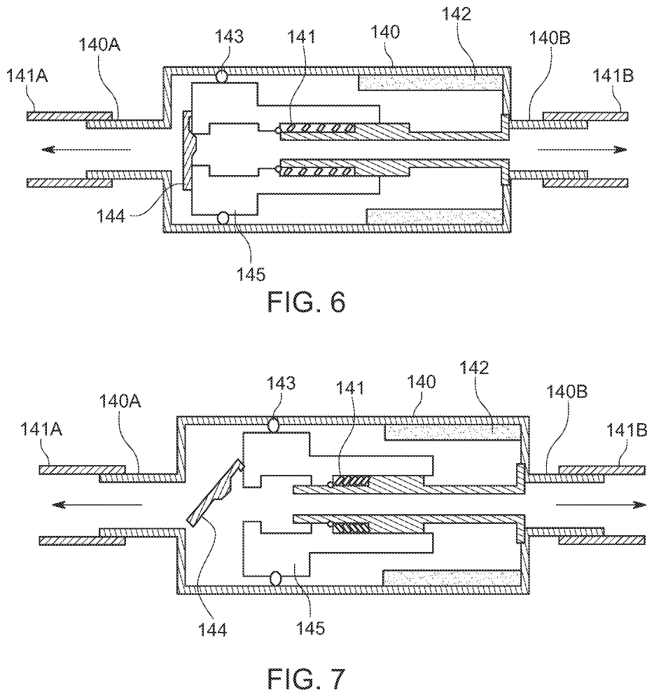

FIGS. 6-8 illustrate an example embodiment of an aspiration system that may apply a near constant, steady state vacuum pressure to a tubing kit and catheter system to aspirate thrombus into a collection canister, with a device, further described below, being connected as part of the tubing kit between the collection canister and the catheter. Such an example embodiment may be utilized to selectively apply a surge pressure level to aid in breaking up a clot that has become stuck in the catheter.

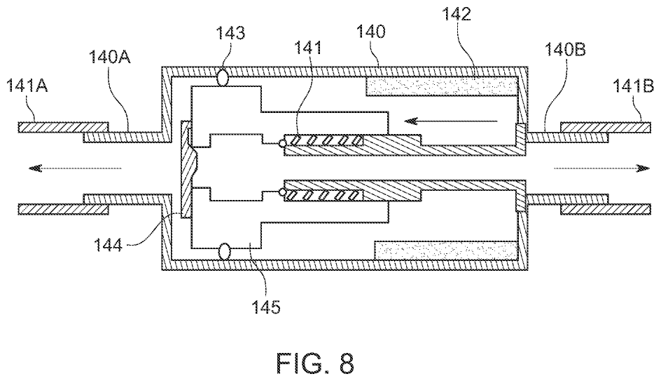

FIGS. 6-8 illustrate a full cycle of an example embodiment of the static aspiration system. FIG. 6 illustrates a first operational state, with the plunger in its starting position and the valve being closed. FIG. 7 illustrates a second operational state, in which the plunger has retracted to open the valve. FIG. 8 illustrates the plunger having returned to its original position to close the valve, thus returning to the first operational state.

As shown in FIGS. 6-8, a cylinder 140 may function as a housing for various internal components which, working together, may be operable to apply a near constant, steady state vacuum pressure. While the figures illustrate a cylinder 140, it should be appreciated that the shape of the housing may vary in different embodiments. Generally, the cylinder 140 will include an internal cavity in which various components are stored as described below.

The cylinder 140 may also include a first port 140A, which may be in communication with a pressure source including a canister, and a second port 140B, which may be in communication with a catheter 100. In an example embodiment as shown in FIG. 6, the first port 140A of the cylinder 140 may be fluidly connected to a corresponding first conduit 141A, with the first conduit 141A being connected to a pump and/or canister for collecting the clot. The second port 140B of the cylinder 140 may be fluidly connected to a corresponding second conduit 141B, with the second conduit 141B being connected to a catheter 100.

The internal cavity of the cylinder 140 may include a plunger 145 as shown in the figures. The plunger 145 may comprise a paramagnetic material that may slide back and forth within the cylinder 140 such as shown in FIGS. 6-8. The shape, size, and configuration of the plunger 145 may vary in different embodiments, and thus should not be construed as limited by the example embodiment shown in the figures. One or more O-rings 143 may be positioned between an outer edge of the plunger 145 and an inner wall of the cylinder 140 so as to provide a seal between the plunger 145 and the cylinder 140.

As shown throughout FIGS. 6-8, a valve 144 may be attached to the plunger 145 so as to selectively seal a passageway extending through the plunger 145. The valve 144 may in an example embodiment comprise a polymeric flap valve such as shown in the figures. The valve 144 may be hingedly attached to the plunger 145 such that the valve 144 may be hingedly adjusted between a first position which seals the passageway through the plunger 145 and a second position which at least partially opens the passageway through the plunger 145.

The valve 144 may be opened or closed through adjustment of the plunger between two or more positions. Generally, the plunger 145 may be adjustable between a first position, in which the valve 144 encloses the passageway through the plunger 145 as shown in FIG. 6, and a second position, in which the valve 144 is opened to unseal the passageway through the plunger 145 as shown in FIG. 7.

The manner by which the plunger 145 is adjusted between its positions may vary in different embodiments. For example, various actuators known in the art, including magnetic, electric, mechanical, and/or electromagnet actuators, may be utilized to adjust positioning of the plunger 145. In an example embodiment as shown in the figures, the plunger 145 may comprise a paramagnetic material and the actuator may comprise a coil 142.

As shown in FIGS. 6-8, the coil 142 may comprise a wire or other elongated member composed of a conductive material which is wound along an inner wall of the cylinder 140. The plunger 145 may be attracted towards the coil 142 when the coil 142 is energized with an appropriate voltage and current, such as is shown in FIG. 7. When the electrical current through the coil 142 is stopped, or lowered to a particular level, the coil 142 may become deenergized and thus release the plunger 145. One or more springs 141 may be operable to return the plunger 145 to its original starting position such as shown in FIG. 8.

Put differently, when the coil 142 is energized, the plunger 145 may overcome the force of the spring 141 and move to its energized position as shown in FIG. 7. A valve 144, which may be movably (e.g., hingedly, axially, rotatably, etc.) connected to the plunger 145, may then open so as to allow fluid to traverse therethrough so as not to affect the overall pressure applied by an associated pressure source such as shown in FIG. 7.

When the coil 142 is deenergized, the plunger 145 may revert back to its original force due to force exerted by the spring 141. The back pressure of the fluid acting on the outside of the valve 144 may then result in the valve 144 being closed again such as shown in FIG. 8. With the valve 144 closed, the return stroke of the plunger 145 may create an additional suction pressure over the static pressure being applied by an associated pump, and thereby a surge vacuum pressure may be applied, such as for breaking up a clot.

Energizing and deenergizing the coil 142 successively may thus result in the pressure cycling between the baseline static vacuum pressure generated by the pump and the surge pressure created by the movement of the plunger; thereby achieving dynamic aspiration.

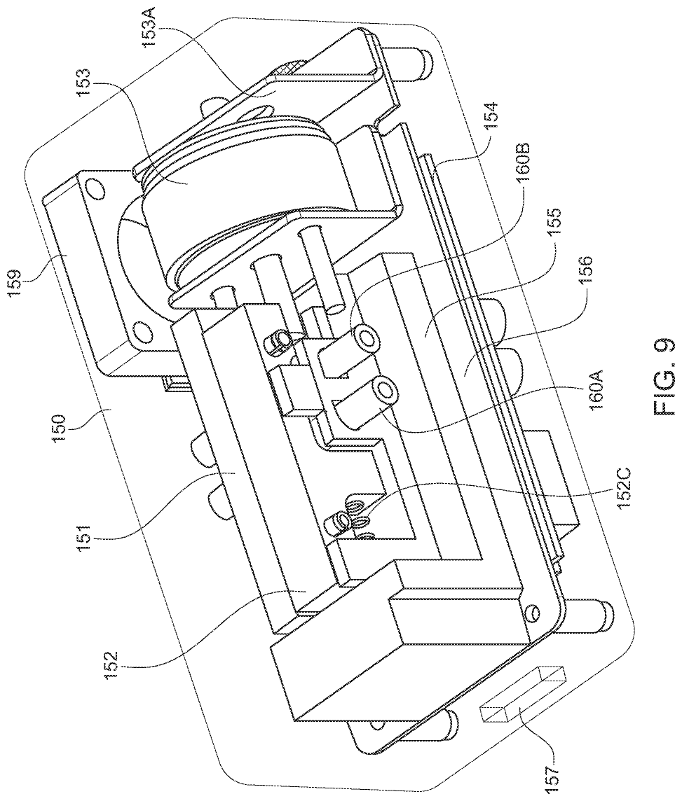

FIGS. 9-11 illustrate yet another example embodiment of an aspiration thrombectomy system. The example embodiment of FIGS. 9-11, like the other example embodiments shown and/or described herein, may be configured to facilitate a non-linear, dynamic thrombectomy using a novel approach wherein a disposable module, provided in a terminally sterilized state as part of an aspiration tubing kit, provides the ability to aspirate a clot using either a linear vacuum pressure profile or various non-linear profiles.

Continuing to reference FIGS. 9-11, an example embodiment may comprise a tubing kit that, when connected to an aspiration pump, may provide the operator with the ability to use non-linear vacuum pressure profiles to aspirate a thrombus. Such an example embodiment may take the form of a disposable tubing kit with an electronic module allowing an operator to switch between static aspiration to a choice of non-linear vacuum pressure profiles.

The module may comprise a top and bottom enclosure including a valve body sub-assembly, a solenoid, a circuit board, a cooling fan, brackets, and bolster plates to secure everything together. A selector switch may enable the operator to switch between static aspiration and dynamic aspiration, or to select a particular dynamic vacuum pressure profile.

The module may be powered by an aspiration pump. A communication port may enable both the transmittance of power from the pump as well as two-way communication of signals between the circuit board of the module (if included) and circuitry in the pump. However, in some example embodiments as described below, the circuit board may alternatively reside in the pump itself.

The valve body may allow for tubing from two different vacuum sources (e.g., a first source at a deep vacuum pressure and a second source at a higher pressure than the first source, which may be referred to as a “relief pressure”). The tubing may enter the module from the pump side and exit through the patient side to converge, such as through a Y-shaped connector, before connecting to an aspiration catheter.

In the example embodiment shown in FIGS. 9-11, it can be seen that an enclosure 150 may function as a housing for storing the various internal components of the system. The shape, size, and configuration of the enclosure 150 may vary in different embodiments, and thus should not be construed as limited in scope by the example embodiments shown in the figures.

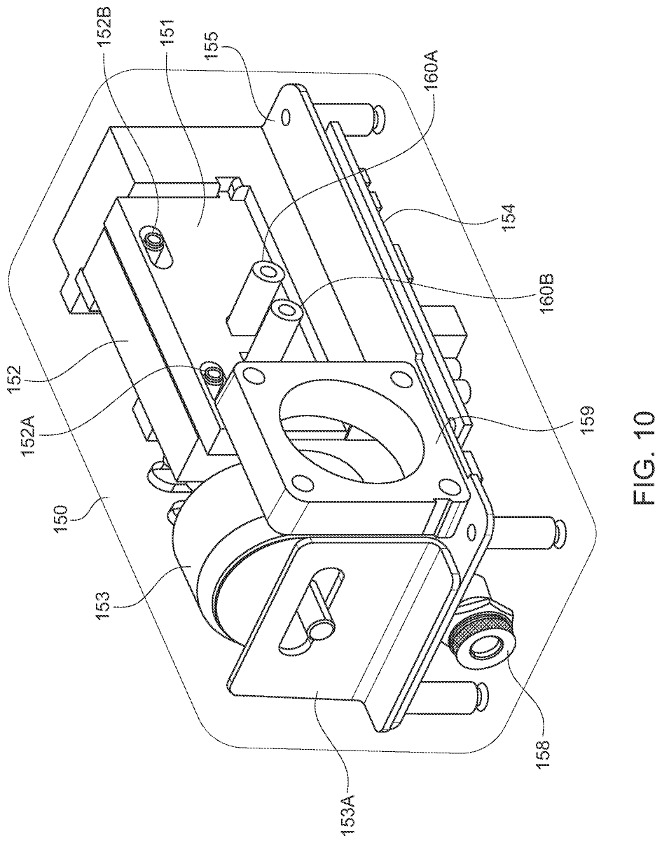

An interior of the enclosure 150 may include the various components of the aspiration system. As shown in FIGS. 9 and 10, a bolster plate 156 may be fixed within the enclosure 150, such as by various types of fasteners. A bed 155 may be fixed to the bolster plate, with the bed 155 including both a vertical portion, which may function as a stopper for a slide 152, and a horizontal portion, on which a valve body 151 may be positioned. Thus, the bed 155 may comprise a substantially L-shaped configuration such as best shown in FIG. 9.

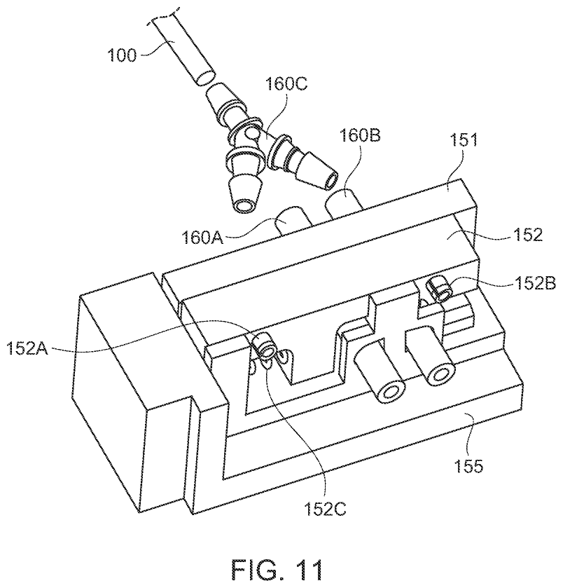

As best shown in FIG. 11, it can be seen that a valve body 151 may be shaped and sized to receive a slide 152 thereon. A pair of conduits 160A, 160B may extend through both the valve body 151 and the slide 152. A first conduit 160A may be connected between a first pressure source 110A (e.g., vacuum pressure) and a catheter 100 and a second conduit 160B may be connected between a second pressure source 110B (e.g., relief pressure) and a catheter 100, with both the first and second conduits 160A, 160B passing through the valve body 151 to be selectively and alternatively sealed by the slide 152 as discussed herein.

Various types of conduits 160A, 160B may be utilized, including but not limited to various types of pinch tubing known in the art. Further, various types of pressure sources 110A, 110B may be utilized. Generally, the pressure sources 110A, 110B may rely upon air or inert gas, rather than any fluids. Both pressure sources 110A, 110B may be comprised of a single aspiration pump. In an example embodiment, one of the pressure sources 110A, 110B may comprise ambient atmosphere.

Continuing to reference FIG. 11, it can be seen that a slide 152 may be movably connected to the valve body 151 such that the slide 152 may adjust between a first position, in which the slide 152 seals the first conduit 160A, and a second position, in which the slide 152 seals the second conduit 160B. In this manner, the level of pressure applied to the catheter 100 may be alternated between a first (deep vacuum) pressure and a second (relief) pressure, which is higher than the first pressure.

The manner by which the slide 152 is adjusted between its positions may vary in different embodiments. Generally, an actuator may be utilized to adjust the slide 152 in at least one direction (e.g., from its first position to its second position). As a non-limiting example, the actuator may be operable to push and/or pull the slide 152. Various types of actuators known in the art may be utilized, including electrical, mechanical, magnetic, and/or electromagnetic actuators.

In an example embodiment as shown in FIGS. 9-10, it can be seen that the actuator may comprise a solenoid 153. When the solenoid 153 is energized, the slide 152 may be actuated forward so as to seal, e.g., by pinching, one of the two conduits 160A, 160B. More specifically, in an example embodiment, energizing the solenoid 153 may move the slide 152 to engage with and seal the second conduit 160B, while the first conduit 160A remains open.

Continuing to reference FIGS. 9-10, it can be seen that the solenoid 153 may be mounted to a bracket 153A that itself may be secured to the bed 155 within the enclosure 150. The bracket 153A may include an opening or slot through which a shaft of the solenoid 153 may extend such as shown in FIG. 10. A cooling device, such as a cooling fan 159, may be positioned adjacent to the solenoid 153 for cooling purposes. The enclosure 150 may include corresponding vent openings to accommodate airflow from the fan 159.

Generally, the slide 152 may be biased towards the first position in which the deep vacuum pressure is applied. Thus, absent application of force from an actuator such as a solenoid 222, the slide 152 may rest in a position in which the first conduit 160A is sealed and the second conduit 160B is opened. Various types of biasing members may be utilized to bias the slide 152 towards the first position. In an example embodiment as shown in FIGS. 9-10, it can be seen that a spring 152C may bias the slide 152 towards the first position such that, absent force from an actuator such as the solenoid 153, the slide 152 naturally may revert back to its original position. Various types of springs 152C may be utilized, such as but not limited to coil springs, compression springs, torsion springs, and the like.

As best shown in FIG. 10, the slide 152 may include slide pins 152A, 152B which rest and ride within corresponding slots formed in the valve body 151. The slide pins 152A, 152B may function to guide the slide's 152 movement and act as stoppers to prevent the slide 152 from moving too far in either direction. In the example embodiment shown in FIG. 10, a first slide pin 152A is slidably positioned within a first slot of the valve body 151 and a second slide pin 152B is slidably positioned within a second slot of the valve body 151. It should be appreciated, however, that in other embodiments, more (e.g., three or more) or less (e.g., one) slide pins 152A, 152B may be utilized.

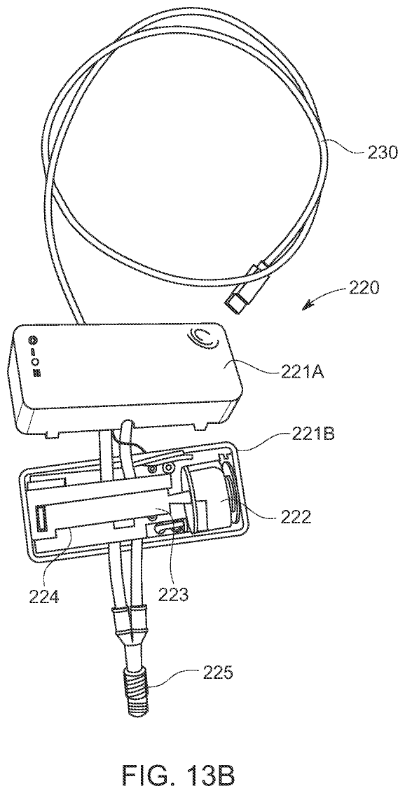

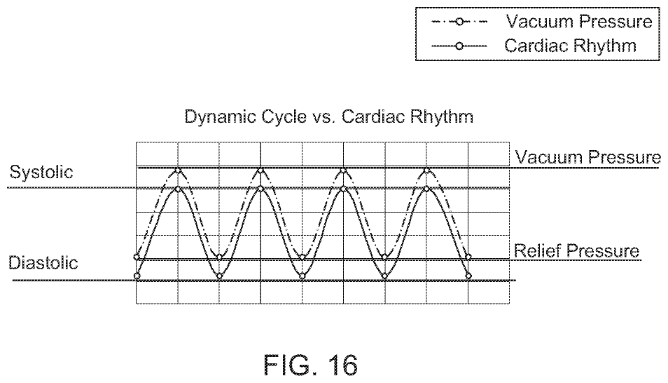

With reference to FIGS. 9-10, it can be seen that a circuit board 154 may be positioned underneath the bed 155. The circuit board 154 may control operation of the solenoid 153 and thus, by extension, the slide 152. The circuit board 154 may be configured to direct movement of the slide 152 at certain times, such as to follow cardiac rhythms as discussed in more detail below and shown in FIG. 16. In some example embodiments, the circuit board 154 may instead be integrated with the pump console 200, rather than the aspiration module 220, as is discussed below and shown in FIG. 13A.

As best shown in FIG. 9, a selector switch 157 may be positioned on a side of the enclosure 150. It should be appreciated that the positioning of the selector switch 157 on the enclosure 150 may vary in different embodiments, and thus should not be construed as limited in scope by the example embodiment shown in the figures. The selector switch 157 may be utilized to manually select different pressure levels to be applied to the catheter 100.

As best shown in FIG. 10, a communication port 158 may be positioned on a side of the enclosure 150. It should be appreciated that the positioning of the communication port 158 on the enclosure 150 may vary in different embodiments, and thus should not be construed as limited in scope by the example embodiment shown in the figures. The communication port 158 may be in communication with the circuit board 154 and/or solenoid 153.

In use, the example embodiment shown in FIGS. 9-11 may be first connected between one or more pressure sources 110A, 110B and a catheter 100. A first end of each of the conduits 160A, 160B may be fluidly connected to the respective pressure sources 110A, 110B, such as to a pump console 200. A second end of each of the conduits 160A, 160B may be fluidly connected to the catheter 100, such as through use of a Y-connector 160C such as a Y-joint as shown in FIG. 11. The catheter 100 may then be advanced to the site of a thrombus and the pump console 200 may be activated to apply a vacuum pressure to the catheter 100. The pressure level may be adjusted as needed by manually using the selector switch 157, or automatically through programming of the circuit board 154, to aspirate the thrombus into the catheter 100 and eventually to be collected, such as within a canister.

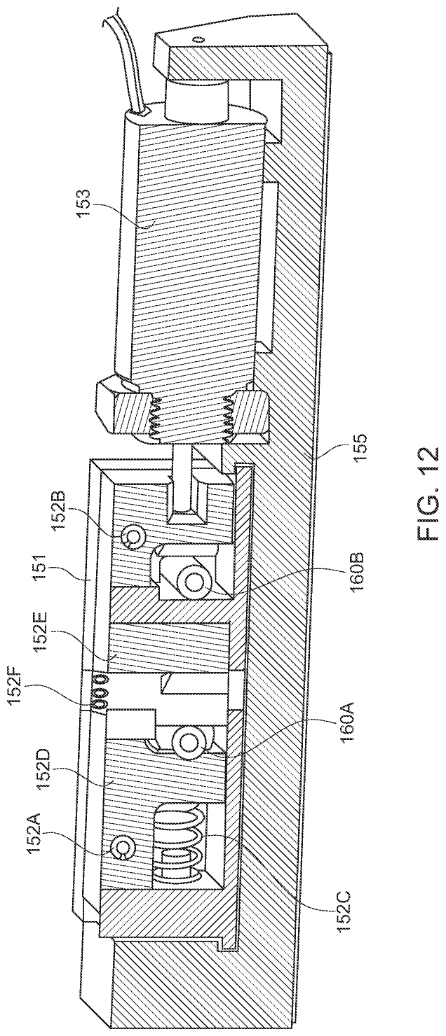

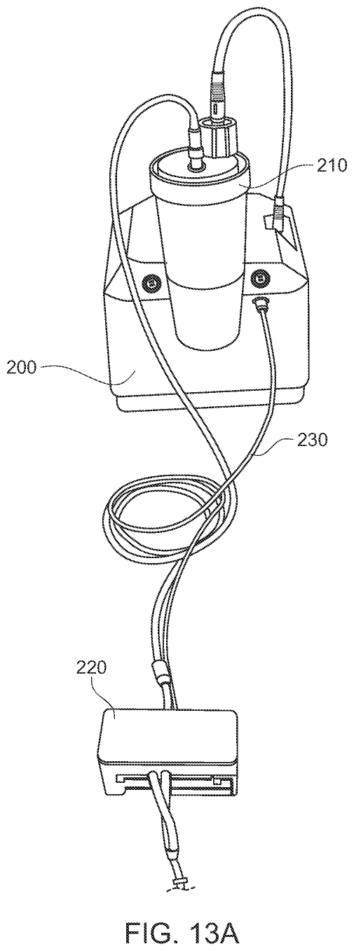

It should be appreciated that the example embodiment shown in FIGS. 9-11 may have moments in time where both conduits 160A, 160B are opened, such as when the slide 152 is positioned between the conduits 160A, 160B without sealing either. FIG. 12 illustrates an example embodiment which ensures that only one conduit 160A, 160B is open at any given time.

In the example embodiment shown in FIG. 12, it can be seen that the slide 152 is split between a first slide portion 152D and a second slide portion 152E. A biasing member such as a slide spring 152F may be positioned between the slide portions 152D, 152E. In this manner, any “dead zone” between the two conduits 160A, 160B may be eliminated such that one of the conduits 160A, 160B is always sealed and one of the conduits 160A, 160B is always opened, with no period of time in which both conduits 160A, 160B are opened or closed simultaneously.

FIGS. 13A and 13B illustrate an overall aspiration system including a pump console 200 and a dynamic aspiration module 220. Various types of pumps and pump consoles known in the art may be utilized. The dynamic aspiration module 220 may comprise any of the example embodiments of aspiration systems described and/or shown herein.

FIG. 13A illustrates interconnection between the pump console 200 and an example embodiment of a dynamic aspiration module 220. In such an example embodiment, the circuit board 154 may be integrated with the pump console 200, rather than the aspiration module 220, such that control is performed by the pump console 200. A communication cable 230 may be electrically connected between the pump console 200 and the aspiration module 220 so as to control operation of the aspiration module 220.

The pump console 200 may include a vacuum canister 210 for receiving and storing the clot after it has been retrieved through the catheter 100. Appropriate conduits may be fluidly connected between the pump console 200 and the aspiration module 220 before eventually converging into the catheter 100.

FIG. 13B provides a closer view of an example embodiment of the dynamic aspiration module 220 shown in FIG. 13A. However, as previously mentioned, any of the systems and/or methods shown and/or described herein for dynamic and/or steady aspiration may be utilized with the pump console 200 of FIG. 13A.

In the example embodiment shown in FIG. 13B, it can be seen that the enclosure 150 may comprise a top housing 221A and a bottom housing 221B which may be attached together to form the completed enclosure 150. A solenoid 222 and slide mechanism 223 may be positioned within the enclosure 150 between the top and bottom housings 221A, 221B above a bolster plate 224. A catheter port 225 may be provided after convergence of the conduits 160A, 160B to which the catheter 100 may be fluidly connected. A communication cable 230 may extend from the enclosure 150 to be connected to the pump console 200.

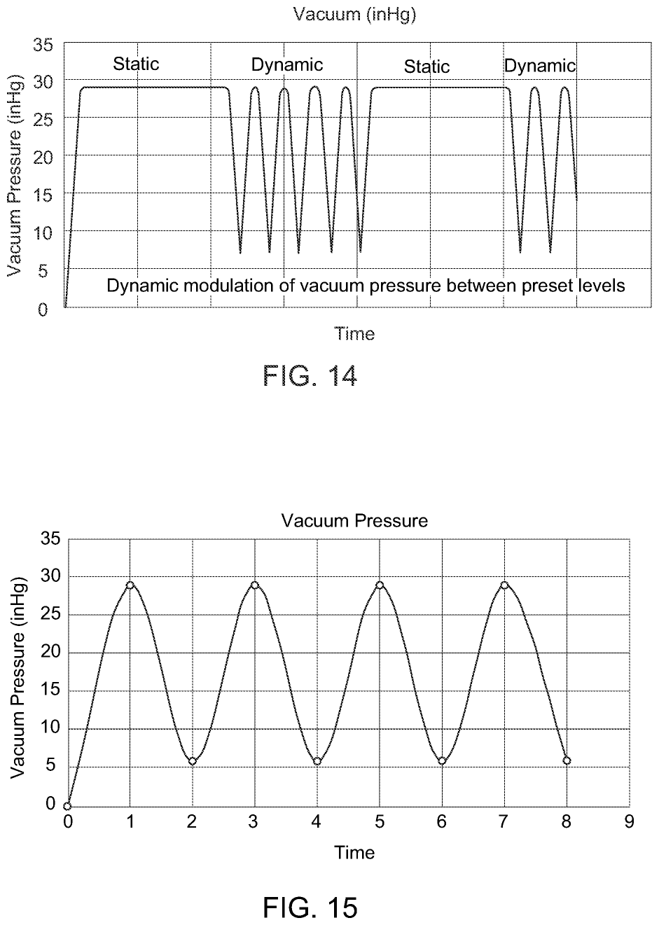

FIGS. 14-16 illustrate example pressure profiles which may be effectuated using the various systems and/or methods shown and/or described herein. FIG. 14 illustrates a pressure profile which alternates between a static pressure, in which a constant vacuum level is applied to the catheter 100, and a dynamic pressure, in which alternating high and low vacuum levels are applied to the catheter 100 to produce a “jack hammer” effect, such as when needed to break up a clot that has become stuck in a catheter 100. FIG. 15 illustrates a closer look at a dynamic pressure, showing how the pressure may alternate between a higher pressure and a lower pressure to produce the “jack hammer” effect.

In some example embodiments, the aspiration system may be configured to apply dynamic pressure levels which correlate closely with a cardiac rhythm of a patient. FIG. 16 illustrates example pressure profiles in which the pressure levels are synced with a cardiac rhythm of the patient.

The cyclic frequency of the pressure levels may be phased/synced such that relief pressure is applied between beats (diastolic) and vacuum pressure is applied in sync with the beats (systolic). Such a configuration may ensure that during the relief cycle, where pressure on the clot is loosened ever so slightly, the systolic pressure of the cardiac rhythm does not carry the clot away from the catheter 100.

In an example embodiment, data from a cardiac monitor such as a heart rate monitor may be communicated to the control circuitry of the aspiration system, such as to a circuit board 154 which is integrated with either the pump console 200 or the aspiration module 220 as described above. However, various other methods may be utilized to sync the pressure profile with the cardiac rhythm of the patient, including but not limited to manual switching by use of the selector switch 157. As another example, a pressure sensor, such as a localized pressure sensor, may be utilized for syncing with the cardiac rhythm of the patient.

### Clauses

Exemplary embodiments are set out in the following numbered clauses:

Clause 1. A method of removing a thrombus from a patient may comprise applying a first pressure level to an aspiration catheter when retrieving a clot and, if the clot becomes stuck in the catheter, applying a second pressure level to break up the clot.

Clause 2. A method of removing a thrombus from a patient may comprise applying a steady pressure profile to an aspiration catheter to retrieve the clot.

Clause 3. A method of removing a thrombus from a patient may comprise applying a dynamic pressure profile to an aspiration catheter to break up the clot, the dynamic pressure profile including alternating between a higher pressure level and a lower pressure level.

Clause 4. A method of removing a thrombus from a patient may comprise alternating between a steady pressure profile and a dynamic pressure profile when aspirating a clot.

Clause 5. A method of removing a thrombus from a patient may comprise biasing a valve or other mechanism such that, absent power or application of force, a steady suction level is still applied to the aspiration catheter.

Clause 6. A method of removing a thrombus from a patient may comprise monitoring a cardiac rhythm of a patient and syncing a dynamic pressure profile with the cardiac rhythm.

Clause 7. A method of removing a thrombus from a patient may comprise applying a first vacuum level between cardiac beats and applying a second vacuum level in sync with the cardiac beats.

Clause 8. A method of removing a thrombus from a patient may comprise connecting a cartridge between an aspiration pump and an aspiration catheter, with a first conduit connected to a first pressure source and a second conduit connected to a second pressure source being routed through the cartridge.

Clause 9. A method according to clause 8 may comprise actuating a first hammer to seal the first conduit and actuating a second hammer to seal the second conduit.

Clause 10. A method of removing a thrombus from a patient may comprise connecting a cylinder between an aspiration pump and an aspiration catheter, the cylinder including a paramagnetic plunger configured to adjust between a first position, in which a steady vacuum level is applied to the catheter, and a second position, in which a higher, surge vacuum level is applied to the catheter.

Clause 11. A method according to clause 10 may comprise hingedly attached a valve so as to selectively seal a passage extending through the plunger such that, when the plunger is adjusted to the second position, the valve is opened to expose to the passage.

Clause 12. A method of removing a thrombus from a patient may comprise adjusting a sliding mechanism between a first position in which the sliding mechanism seals a first conduit connected to a first pressure source applying a first pressure level and a second position in which the sliding mechanism seals a second conduit connected to a second pressure source applying a second pressure level.

Clause 13. A method according to clause 12 may comprise actuating the sliding mechanism with a solenoid.

Clause 14. A method according to clauses 12 and/or 13 may comprise biasing the sliding mechanism towards a first position.

It should be noted that any of the embodiment, features, or details of this specification can be used in connection with each other. In other words, while specific features may have been described separately, it is contemplated that any combination of these features can be combined with each other. Hence, this specification includes embodiments with any combination of the features described herein.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. An aspiration thrombectomy system, comprising:
a catheter;
a first pressure source;
a second pressure source;
a first conduit connected between the catheter and the first pressure source;
a second conduit connected between the catheter and the second pressure source; and
a sealing mechanism;
wherein in a first configuration the sealing mechanism seals the second conduit such that the first pressure source applies a first pressure to the catheter;
wherein in a second configuration the sealing mechanism seals the first conduit such that the second pressure source applies a second pressure to the catheter; and,
wherein the second pressure is higher than the first pressure.

2. The aspiration thrombectomy system of claim 1, wherein the first pressure is between 508-762 mmHg below ambient pressure.

3. The aspiration thrombectomy system of claim 2, wherein the second pressure is between 0-508 mmHg below ambient pressure.

4. The aspiration thrombectomy system of claim 1, wherein the sealing mechanism is comprised of a sliding member which is slidable between the first configuration and the second configuration.

5. The aspiration thrombectomy system of claim 4, wherein in the first configuration the sliding member pinches the first conduit, and wherein in the second configuration the sliding member pinches the second conduit.

6. The aspiration thrombectomy system of claim 1, wherein the sealing mechanism is biased towards the first configuration.

7. The aspiration thrombectomy system of claim 1, further comprising a spring for biasing the sealing mechanism towards the first configuration.

8. The aspiration thrombectomy system of claim 1, further comprising an actuator for adjusting the sealing mechanism between the first configuration and the second configuration.

9. The aspiration thrombectomy system of claim 8, wherein the actuator is comprised of a solenoid.

10. The aspiration thrombectomy system of claim 1, wherein the first pressure source and the second pressure source are comprised of a single pump.

11. The aspiration thrombectomy system of claim 1, wherein the first conduit and the second conduit are connected to the catheter by a Y-joint.

12. The aspiration thrombectomy system of claim 1, wherein in a first operational state, a static pressure is applied to the catheter, and wherein in a second operational state, a dynamic pressure is applied to the catheter.

13. The aspiration thrombectomy system of claim 12, further comprising a switch for selecting between the first operational state and the second operational state.

14. The aspiration thrombectomy system of claim 1, further comprising a heart rate monitor in communication with the sealing mechanism such that the sealing mechanism adjusts between the first configuration and the second configuration in sync with a cardiac rhythm of a patient.

15. An aspiration thrombectomy system, comprising:
a catheter;
a pump;
a first conduit connected between the catheter and the pump;
a second conduit connected between the catheter and the pump; and
a sealing mechanism;
wherein in a first configuration the first conduit applies a first pressure to the catheter and the second conduit is sealed by the sealing mechanism; and,
wherein in a second configuration the second conduit applies a second pressure to the catheter and the first conduit is sealed by the sealing mechanism.

16. The aspiration thrombectomy system of claim 15, wherein the second pressure is greater than the first pressure.

17. The aspiration thrombectomy system of claim 16, wherein the first pressure is between 508-762 mmHg below ambient pressure and wherein the second pressure is between 0-508 mmHg below ambient pressure.

18. The aspiration thrombectomy system of claim 15, wherein the sealing mechanism is comprised of a sliding member which is slidable between the first configuration and the second configuration.

19. The aspiration thrombectomy system of claim 15, further comprising a solenoid for adjusting the sealing mechanism between the first configuration in which the sealing mechanism seals the second conduit and the second configuration in which the sealing mechanism seals the first conduit.

20. An aspiration thrombectomy system, comprising:
a catheter;
a pumping means for applying a first pressure or a second pressure to the catheter;
a first conduit connected between the catheter and the pumping means;
a second conduit connected between the catheter and the pumping means; and
a sealing means for selectively sealing the first conduit or the second conduit; and,
wherein the sealing means is biased towards sealing the first conduit.
